(19) United States
(12) Patent Application Publication
(10) Pub. No.: US 2011/0015668 A1
(43) Pub. Date: Jan. 20, 2011

(54) ELASTIC VENOUS COMPRESSION STOCKING THAT IS EASIER TO PUT ON

(76) Inventor: Francois Cros, Ivry Sur Seine (FR)

Correspondence Address: JACOBSON HOLMAN PLLC, 400 SEVENTH STREET N.W., SUITE 600, WASHINGTON, DC, 20004, US

(21) Appl. No.: 12/736,302

(22) PCT Filed: Mar. 25, 2009
(86) PCT No.: PCT/FR2009/000324

§ 371 Date: Sep. 28, 2010

(30) Foreign Application Priority Data
Mar. 28, 2008 (FR) 0801692

(51) Int. Cl. A61B 17/00

(52) U.S. Cl. 606/201

(57) ABSTRACT

The orthosis includes a compressive portion having an ankle portion that, when worn, exerts a nominal textile pressure P₀ for an ankle circumference C₀. When it is stretched in order to enable it to go past the instep region of the leg, the ankle portion exerts an elastic return force in the circumferential direction corresponding to a nominal putting-on textile pressure P₁ for an instep circumference C₁. At least one parameter selected from: the natures of the core yarns used for said knit yarn and said weft yarn; any covering of said yarns; and the knitting of said yarns; is selected in such a manner that for C₁=1.38C₀, said nominal putting-on textile pressure P₁ is less than or equal to 120% of said nominal wearing pressure P₀. This makes it easier for the patient to put on and take off the orthosis merely as a result of the intrinsic properties thereof and without having recourse to any accessory.

The invention relates to elastic venous compression (EVC) orthoses that are indicated in various clinical manifestations of venous insufficiency of the lower limbs.

Such orthoses, once known as “compression stockings” or “compression socks” are medical textile devices producing a therapeutic effect by applying a compression gradient to the lower members, in contrast to “support stockings” (or “anti-embolism stockings” or “aching-leg stockings” and “fashion stockings”, which are not medical devices with a therapeutic purpose.

EVC orthoses are designed to produce a therapeutic effect by compressing the lower limb over a greater or lesser extent, with pressure P₀ measured at the ankle lying in the range 10 millimeters of mercury (mmHg) to more than 36 mmHg (i.e. 13 hectopascals (hPa) to 48 hPa, it being understood that mmHg is nevertheless the unit commonly used to measure pressure in the field of phlebology and medical compression). These orthoses are subdivided into four textile classes in application of the French ASQUAL scale, i.e. class I (13 hPa to 20 hPa, i.e. P₀≈10 mmHg to 15 mmHg at the ankle), class II (20 hPa to 27 hPa, i.e. P₀≈15 mmHg to 20 mmHg), class III (27 hPa to 48 hPa, i.e. P₀≈20 mmHg to 36 mmHg), and class IV (>48 hPa, i.e. P₀>36 mmHg).

These orthoses generally provide compression that decreases going upwards in the portion situated above the ankle, as indicated incidentally in FR 2 755 006 A1 (Couzan et al.), sometimes with a profile that is specially adapted with the pressure maximum being offset above the ankle, as described in WO 2006/134250 A2 (Innothéra Topic International).

It should be observed that other scales also exist (e.g. RAL GZ 387 in Germany) or other practices that specify compression classes differently, without thereby changing the function of this type of product.

In order to enable a high level of compression on the lower limbs, such orthoses are made from a knitting stitch having a texture that is made firm to a greater or lesser extent with an incorporated elastic weft yarn, generally covered in spandex. They are also closely dimensioned as a function of the patient's limb in order to obtain the desired degree of pressure and pressure profile by elasticity.

This gives rise in particular to a difficulty in putting the orthosis on and in taking it off, in particular in the region of the instep and the heel, particularly when the textile class of the orthosis is high.

This difficulty in putting on is generally made worse by the fact that such orthoses are prescribed for treating venous troubles suffered by patients who are elderly, clumsy, sometimes suffering motor handicap, suffering arthroses that deform the hands and the feet, etc., i.e. patients generally having limited mobility.

This difficulty of putting compression orthoses on is an obstacle that is known to patients and carers, and numerous types of accessory have been proposed for this purpose.

Such accessories are generally in the form of a metal structure onto which the orthosis is initially engaged so as to stretch it and thus make it easier to insert the patient's foot and ankle. Nevertheless, they are all more or less difficult to use, which explains their lack of success with patients.

One of the objects of the invention is to propose a compressive orthosis that mitigates those difficulties and that is easier for the patient to put on and take off, merely by means of its intrinsic properties and without having recourse to any stretching accessory.

The starting point of the invention is the observation that the difficulty occurs mainly when the orthosis is stretched to the maximum, i.e. when the patient needs to pass it over the region situated between the instep and the heel (referred to below as the “instep region”, where the instep is defined as being the anterior top portion situated in the region where the foot is curved). Specifically:
for the orthosis, it is in its ankle portion:
   that firstly the circumference of the orthosis is at its smallest (since morphologically, it is at the level of the ankle that the limb is at its narrowest); and
   that secondly the orthosis generally exerts its greatest pressure (given the requirements for pressure to be degressive); and
for the limb, it is the instep region that presents the right section having the greatest circumference.

In other words, in order to be able to move the orthosis past the instep region while it is being put on, it is necessary for the orthosis to be stretched very strongly in its portion that is the narrowest and that applies the greatest compression (the ankle portion).

The invention proposes modifying the rheology of the orthosis, i.e. its elasticity characteristic (pressure/circumference characteristic) in this ankle portion so as to make it easier to put on.

Essentially, the invention proposes modifying this rheology so as to enable this portion to be stretched to a circumference that is as large as that of the instep (much greater than the circumference of the ankle which corresponds to the permanent or “wearing” configuration of the orthosis) without any need for excessive pressure or the help of any accessory.

More precisely, the orthosis of the invention includes a compressive portion extending upwards that, once the orthosis has been put into place on the limb, exerts an elastic return force in the circumferential direction that is suitable for producing compression of the limb at a therapeutic pressure level. This compressive portion includes an ankle portion that may take the following states in succession:
a free state in which the ankle portion is not deformed elastically;
a nominal stretched state on passing the instep region while the orthosis is being put into place on the limb, in which state the ankle portion is elastically deformed to the circumference C₁ of the instep of the limb and exerts an elastic return force in the circumferential direction that corresponds to a nominal putting-on textile pressure P₁; and
a wearing state when the orthosis is in place on the limb in order to produce compression thereon at a therapeutic pressure level, in which state the ankle portion is elastically deformed to the circumference C₀ of the ankle of the limb and exerts an elastic return force in the circumferential direction that produces a nominal wearing textile pressure P₀.

In a manner characteristic of the invention, at least one of the parameters selected from: the natures of the core yarns used for said knit yarn and said weft yarn; any covering of said yarns; and the knitting of said yarns; is selected in such a manner that for C₁=1.38C₀, said nominal putting-on textile pressure P₁ is less than or equal to 120% of said nominal wearing pressure P₀.

In other words, these parameters are selected so as to keep the elastic pressure produced in the stretched state down to a value that is well below that which is encountered in traditional EVC orthoses.

In particular, the ankle and instep circumferences C₀ and C₁ are those of a leg-model as defined by French standard NF G 30-102b, Appendix B, or of a Hohenstein type leg-model in accordance with German scale RAL-GZ 387.

The ankle portion may also take on a maximum stretched state on passing the instep region while the orthosis is being put into place on the limb, in which state the ankle portion is elastically deformed to a circumference C₂ that is greater than the circumference C₁ of the instep of the limb and exerts an elastic return force in the circumferential direction that corresponds to a maximum putting-on textile pressure P₂. Under such circumstances, at least one parameter selected from: the natures of the core yarns used for said knit yarn and said weft yarn; any covering of said yarns; and the knitting of said yarns; is selected in such a manner that for C₂=1.79C₀, said maximum putting-on textile pressure P₂ is less than or equal to 140% of said nominal wearing textile pressure P₀.

There follows a description of an implementation of the device of the invention with reference to the accompanying drawings.

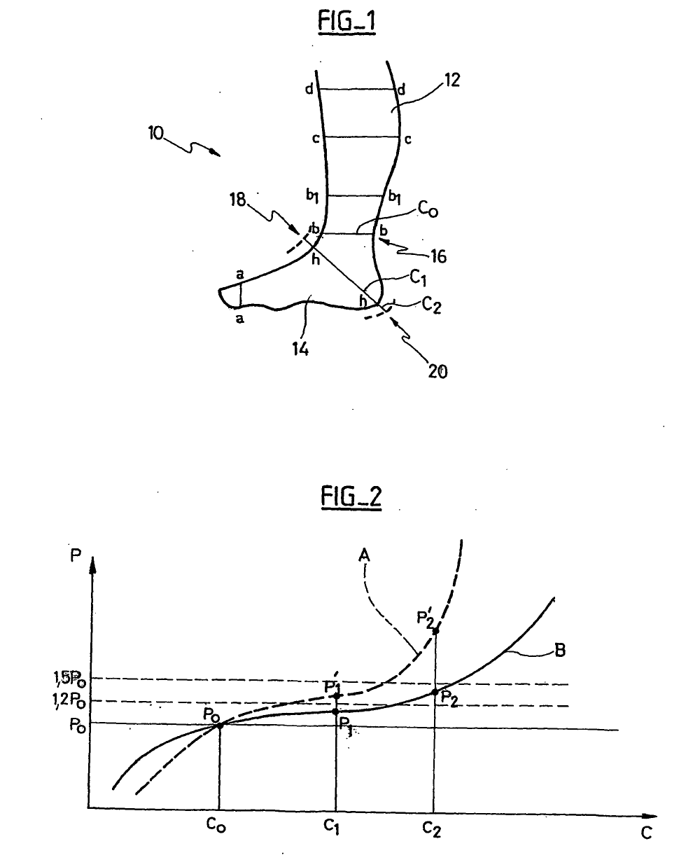

FIG. 1 is a diagrammatic view showing the various zones concerned by the orthosis elasticity profile of the invention.

FIG. 2 plots the elasticity characteristic (pressure/circumference) of an orthosis of the invention.

FIG. 1 shows the various portions of the bottom portion of an orthosis 10 that, in the example shown, is an orthosis that has a closed bottom end covering the foot, e.g. a sock, a stocking, or tights (however the invention is equally applicable to open-footed tights, for example).

These various portions comprise a leg portion 12 extending upwards from the ankle, and an adjacent foot portion 14 extending from the malleolar region to the region of the toes.

The leg portion 12 is a compressive portion with a stitch structure and dimensions that are selected in such a manner as to apply therapeutic pressure to the leg once the orthosis has been put in place on the limb.

Under the effect of being put in place on the limb, the stretch textile exerts a compression effect that results from the return force from elastic fibers that make up the material and from the application of these elastic return forces to the perimeter of the outline at a given point. In application of the Laplace-Young equation, this gives rise to local pressure that is inversely proportional to the radius of curvature of the outline at said point. This pressure is the “textile pressure” as defined and calculated in the meaning of French standard NF G 30-102b.

In the present description, the term “circumference” is used to designate the perimeter of said outline.

The selected stitch and yarn, and the dimensioning of the rows of stitches are defined in such a manner as to apply predetermined pressure at different heights up the leg, e.g. at the height of the ankle, at the start of the calf, over the calf, at the popliteal fossa, etc., all the way up to the top of the thigh for a thigh stocking or tights. These various pressures are defined for each compression class with reference to measurement templates such as the leg-model defined by French standard NF G 30-102b, Appendix B, or the Hohenstein type leg-model according to German scale RAL-GZ 387.

The various corresponding altitudes are conventionally referenced as follows: b (ankle), b1 (start of calf), c (calf), d (popliteal fossa), . . . .

In the context of the invention, the important parameter is the pressure exerted at level b of the limb, i.e. at the smallest perimeter of the ankle. In this region, the orthosis exerts a standardized pressure P₀ for a circumference C₀ around the ankle of the leg-model that is assumed to represent the leg of the patient.

When putting the orthosis into place or removing it, the ankle portion 16 of the orthosis needs to be stretched beyond its nominal circumference C₀ so as to allow it to pass over the region of the limb that is situated between the instep 18 and the heel 20 (referred to below as the “instep region”), i.e. the region of the foot presenting a right section (section h-h) of greater size. The perimeter of this section corresponds to a circumferential dimension C₁, and typically C₁=1.38C₀ (coefficient taken from German scale RAL-GZ 387 for inspecting medical textiles).

In order to take account of practical circumstances of putting the orthosis into place, a circumference C₂ is also defined, and typically C₂=1.79C₀ which corresponds to an elliptical circumference C₁ of major axis lengthened by the width of two thumbs in order to characterize going past the heel with the help of the hands while putting the orthosis into place or removing it.

FIG. 2 shows the elasticity characteristic of the orthosis as measured at the ankle portion 16 (level b-b).

This characteristic gives, as a function of the stretching of the orthosis (stretching measured by the value of its circumference), the corresponding textile pressure. This curve thus represents the rheology of the orthosis under consideration at the heel portion 16.

Characteristic A shows a typical elasticity profile as measured on a conventional orthosis: for the nominal circumference C₀ the corresponding pressure is the wearing pressure or nominal pressure P₀, defining in particular the compression class of the orthosis. For a circumference C₁ (corresponding to going past the instep region) or C₂ (with additional stretching corresponding to the width of two digits), the pressure exerted increases very quickly because of rheological non-linearities of the orthosis, with the corresponding values of the characteristic A being referenced P′₁ and P′₂.

In order to make the orthosis easier to put on and to take off, the invention proposes modifying its rheological properties in the ankle portion, so as to greatly reduce the pressure exerted by said region when the orthosis is stretched, while conserving the same nominal pressure P₀ while being worn.

Difficulties of putting on (and taking off) are for the most part associated with the force that needs to be exerted in order to make the knitted portion corresponding to the ankle zone of the orthosis go past the level of the instep. This difficulty is made that much worse when the pressure at the ankle is high, i.e. for the highest textile pressure classes.

More precisely, it is appropriate:
for the textile pressure C₁ corresponding to the circumference P₁ to be typically less than or equal to 120% of the nominal wearing pressure P₀ (i.e. in other words P₁≦1.2P₀); and
for the textile pressure exerted for the circumference C₂ corresponding to the maximum expected stretching typically to be less than or equal to 140% of the nominal wearing pressure P₀ (i.e. in other words P₂≦1.4P₀).

In FIG. 2, curve B shows a corresponding elasticity profile measured on an orthosis of the invention with example values P₁ and P₂ that satisfy the above-mentioned criteria (P′₁ and P′₂ being the corresponding values for a conventional orthosis that do not satisfy these criteria).

It can be seen that the nominal wearing pressure P₀ is identical for the orthosis of the invention and for the conventional orthosis, since that is a characteristic required by the standard so that the orthosis produces specifically the looked-for therapeutic effect, as a function of the selected compression textile class.

EVC orthoses have thus been made that present the following characteristics for hose in which P₀>20 mmHg (French classes III and IV), i.e. hose for which the problem of putting the hose on arises the most acutely:
stockings made from a weft yarn covered in non-elastic yarn P₁/P₀=1.1 and P₂/P₀=1.2;
socks made from a weft yarn covered with non-elastic yarn: P₁/P₀=1.2 and P₂/P₀=1.4;
stockings made from a weft yarn covered with elastic yarn, or from a non-covered weft yarn: P₁/P₀=1.1 and P₂/P₀=1.2; and
socks made from a weft yarn covered with elastic yarn, or from a non-covered weft yarn: P₁/P₀=1.1 and P₂/P₀=1.2.

The elasticity profile modified in accordance with the invention may be obtained in various ways: by selecting the fibers used for the knit yarn and/or for the weft yarn of the orthosis, by specific covering of said yarns, by specific knitting parameters, or indeed by a combination of these various possibilities.

More precisely, if consideration is given to a conventional orthosis, it may be described as being the result of:
a weft yarn T made up of a core XA and of covering yarns ZA and SA (where S and Z characterize the turn direction of the covering on the core); and
a knit yarn M made up of a core XM and of covering yarns ZM and SM;
these yarns being combined in application of a scheme defined by a set of predefined knitting parameters P.

Such a conventional orthosis gives an elasticity profile at the ankle as represented by the characteristic A in FIG. 2.

In order to obtain an elasticity characteristic as shown at B in FIG. 2, it is possible to proceed in several ways:

a) using the same weft yarn T (core XA and coverings ZA and SA) and combining it with a knit yarn M′ different from M and made up of a core XM′=XM and a covering ZM′=ZM and SM′=SM with a different set of covering parameters and with the same set of knitting parameters P;

b) using the same weft yarn T (core XA and coverings ZA and SA), and combining it with the same knit yarn M (core XM and coverings ZM and SM), but with a set of knitting parameters P′ different from P;

c) using the same weft yarn T (core XA and coverings ZA and SA), and combining it with a knit yarn M′, different from M, having the same core XM, but with coverings Z′M and S′M that are different from ZM and SM, and with the same set of knitting parameters P′.

These examples are not limiting and other ways of obtaining the looked-for profile may be envisaged, as a variant or in addition, in particular by modifying the weft yarn.

The various materials used may in particular be the following: XA=570 decitex (dtex) spandex, SA=Ne 94/1 cotton, XM=44 dtex spandex, ZM=SM=33 dtex polyamide, X′M=17 dtex spandex, Z′M=60 dtex polyamide, and S′M=Nm 1/186 cotton.

Concerning the knitting parameters: P′=stitch height increased by 50% and weft tension decreased by 10%; P″=spindle speed doubled, stretching increased by 50%.

It should be observed that the above values and selections are given purely by way of indication; various adjustments and adaptations may be necessary, but are within the competence of the person skilled in the art. The purpose is to obtain and modify the rheological characteristics of the orthosis in such a manner that the pressure involved when putting it on (when the ankle portion of the orthosis is stretched to allow the instep to pass through) is little greater than the wearing pressure, but without modifying the wearing pressure which determines the looked-for therapeutic effect.

Preferably, when the weft or knit yarns are modified, the modification applies to the entire orthosis and not only to the ankle portion, so that the product presents the same visual appearance and feel over its entire height, without any discontinuity at ankle level.

1. A lower limb compressive orthosis in the form of a stocking, a sock, or tights, obtained by knitting a knit yarn and a weft yarn, the orthosis (10) including a compressive portion (12) extending upwards and suitable, once the orthosis has been put into place on the limb, for exerting an elastic return force in the circumferential direction to produce compression of the limb at a therapeutic pressure level, said compressive portion (12) including an ankle portion (16) suitable for taking up the following states in succession:
a free state in which the ankle portion is not deformed elastically;
a nominal stretched state on passing the instep region while the orthosis is being put into place on the limb, in which state the ankle portion (16) is elastically deformed to the circumference C₁ of the instep (h) of the limb and exerts an elastic return force in the circumferential direction that corresponds to a nominal putting-on textile pressure P₁; and
a wearing state when the orthosis is in place on the limb in order to produce compression thereon at a therapeutic pressure level, in which state the ankle portion (16) is elastically deformed to the circumference C₀ of the ankle (b) of the limb and exerts an elastic return force in the circumferential direction that produces a nominal wearing textile pressure P₀;
which orthosis is characterized in that at least one of the parameters selected from:
the natures of the core yarns used for said knit yarn and said weft yarn;
any covering of said yarns; and
the knitting of said yarns;
is selected in such a manner that for C₁=1.38C₀, said nominal putting-on textile pressure P₁ is less than or equal to 120% of said nominal wearing pressure P₀.

2. The orthosis of claim 1, wherein said ankle and instep circumferences C₀ and C₁ are those of a leg-model as defined by French standard NF G 30-102b, Appendix B, or of a Hohenstein type leg-model in accordance with German scale RAL-GZ 387.

3. The orthosis of claim 1, wherein the ankle portion (16) may also take on the following state:
a maximum stretched state on passing the instep region while the orthosis is being put into place on the limb, in which state the ankle portion is elastically deformed to a circumference C₂ that is greater than the circumference C₁ of the instep (h) of the limb and exerts an elastic return force in the circumferential direction that corresponds to a maximum putting-on textile pressure P₂; and
wherein at least one parameter selected from:
the natures of the core yarns used for said knit yarn and said weft yarn;
any covering of said yarns; and
the knitting of said yarns;
is selected in such a manner that for C₂=1.79C₀, said maximum putting-on textile pressure P₂ is less than or equal to 140% of said nominal wearing textile pressure P₀.
